(19) United States
(12) Patent Application Publication
(10) Pub. No.: US 2024/0269423 A1
(43) Pub. Date: Aug. 15, 2024

(54) RESPIRATORY THERAPY DEVICES

(71) Applicant: SMITHS MEDICAL INTERNATIONAL LIMITED, Ashford (GB)

(72) Inventors: Ombeline Marone-Hitz, Ashford (GB); Jamie Daniel Perkins, Ashford (GB); Stephen James Field, Canterbury (GB); Steven Mark Tupper, Hythe (GB); Mohammad Qassim Mohammad Khasawneh, Canterbury (GB)

(73) Assignee: SMITHS MEDICAL INTERNATIONAL LIMITED, Ashford (GB)

(21) Appl. No.: 18/568,893

(22) PCT Filed: Jul. 6, 2022
(86) PCT No.: PCT/GB2022/000063

§ 371 Date: Dec. 11, 2023

(30) Foreign Application Priority Data
Jul. 15, 2021 (GB) GB2110225.6

(51) Int. Cl. A61M 16/20; A61M 16/00

(52) U.S. Cl. CPC A61M 16/205; A61M 16/0006; A61M 16/208; A61M 2205/3331

(57) ABSTRACT

An oscillatory PEP device has four valve openings (201) to (204) mounted on a support (205) beneath a rocker arm (106) supporting a valve closure (107). The support is rotatable to position a selected one of the valve openings beneath the valve closure and in line with a breathing path between an inlet (103). (104) and an outlet (111). The rocker arm (106) supports a soft-iron pin (108) at one end, which interacts with one of two permanent magnets (212) or (213) of different strengths on a turntable (211) to apply a force tending to pull the arm down and close the valve opening. The turntable (211) can be rotated to position the desired magnet (212) or (213) to interact with the soft-iron pin on the rocker arm.

This invention relates to respiratory therapy devices of the kind having an inlet through which a user breathes, an opening to atmosphere, and a valve mechanism driven by breathing through the device to produce an oscillating resistance to breathing.

Positive expiratory pressure (PEP) devices, that is, devices that present a resistance to expiration through the device, are now widely used to help treat patients suffering from a range of respiratory impairments, such as chronic obstructive pulmonary disease, bronchitis, cystic fibrosis, and atelectasis. More recently, devices that provide an alternating resistance to flow have been found to be particularly effective in that the expiration and vibration combine to stimulate upwards movement of secretions. One example of such devices is sold under the trade mark Acapella (a registered trade mark of Smiths Medical) by Smiths Medical and is described in U.S. Pat. Nos. 6,581,598, 6,776,159, 7,059,324 and 7,699,054. These devices include a valve with a non-linear discharge orifice having a curved, trumpet shape opening that is opened and closed by a conical shape closure member mounted at one end of a rocker arm. An adjustable magnet arrangement provides a force tending to urge the closure member down into sealing engagement with the orifice against the flow of exhaled air through the orifice. This produces an alternating opening and closing of the valve, which in turn produces an oscillatory or vibrating effect in the user's respiratory passages.

The expiratory force that can be exerted by patients varies according to their respiratory capacity. Some patients can only sustain a workflow of less than 10 L/min for 3 seconds or longer, whereas others can sustain workflows of 15 L/min or greater for 3 seconds or longer. In order to provide the most effective therapy across the range of different patients it is necessary to provide a range of different therapy devices having valves of different sizes. It may also be necessary to tailor the magnetic restoring arrangement accordingly. This creates various problems. Hospitals, clinics, and pharmacies need to stock devices across the entire range so that they can provide the appropriate device for all patients. The manufacturer also needs to stock more devices to be able to supply the appropriate device to the customer. Furthermore, as the patient uses the therapy device and his respiration improves, he needs to move to a different device more suitable for his improved lung function.

It is an object of the present invention to provide an alternative respiratory therapy device.

According to the present invention there is provided a respiratory therapy device of the above-specified kind, characterised in that the valve mechanism includes at least two valve members of different sizes that are selectably connected in the breathing path through the device such that the patient can breathe through one or other of the valve members.

The valve mechanism preferably includes a common valve closure, the valve members having openings of different sizes that can be selectively aligned with the valve closure. The valve members may be mounted on a support that is displaceable relative to the valve closure. The support may be rotatable about an axis parallel to the flow path through the valve members. The device may include a magnet arrangement for providing a restoring force to the valve closure tending to urge the valve closure to a closed position. The magnet arrangement may include at least two permanent magnets of different strength that are selectively displaceable into magnetic interaction with a magnetic element on the valve closure. The permanent magnets may be mounted on a turntable, the turntable being rotatable about an axis parallel to the flow path through the valve openings.

A respiratory therapy device according to the present invention will now be described, by way of example, with reference to the accompanying drawing, in which:

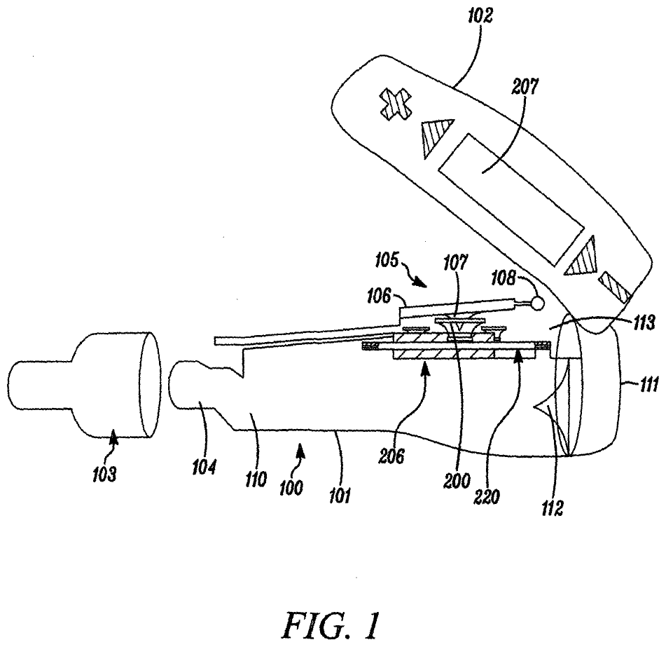

FIG. 1 is a simplified side elevation view of the device with its cover raised; and

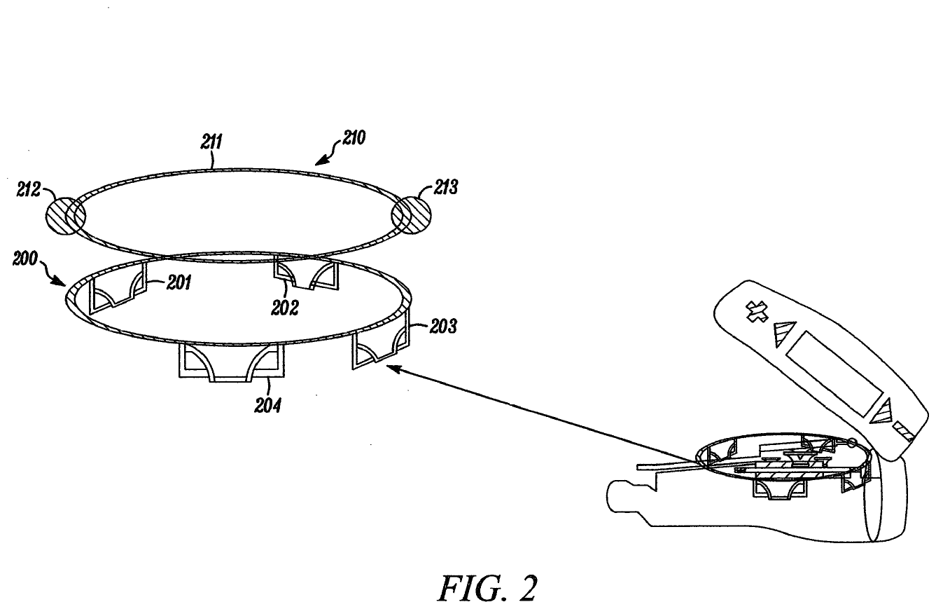

FIG. 2 shows schematically the layout of the valve openings and the magnet arrangement.

With reference first to FIG. 1, the device 100 includes a housing 101 with a normally closed hinged cover 102 and a removable mouthpiece 103. The housing 101 has a patient inlet 104 at its left-hand end to which the mouthpiece 103 is fitted. The housing 101 contains a valve mechanism 105 for producing an oscillating resistance to expiratory flow including a rocker arm 106 pivoted at one end and supporting a conical valve closure 107, similar to that described in U.S. Pat. No. 6,581,598, close to its opposite, free end. The rocker arm 106 also supports a magnetic element 108, such as a soft-iron pin, at its free end. The valve closure 107 faces downwardly, that is, with its pointed tip directed downwardly when the device is held horizontally. The mechanism 105 also includes a valve member or opening providing a trumpet-shape valve seat arrangement indicated generally by the numeral 200 and opening from an air-flow passage 110 extending from the patient inlet 104 to an outlet 111 at the opposite end of the housing 101. The air-flow passage 110 includes a one-way valve 112, such as a duck-bill valve, or flap valve, or some other conventional valve that allows flow in one direction. In particular, the valve 112 allows flow from the outlet 111 to the inlet 104 (during inhalation) but prevents or restricts flow in the opposite direction (during expiration). The valve seat arrangement 200 in the mechanism 105 opens from the air-flow passage 110 into a space 113 above the passage that is normally enclosed, when the cover 102 is in the lower, closed position during use. This space 113 communicates directly with the outlet 111 without the interposition of a one-way valve.

With reference now also to FIG. 2, the valve seat arrangement 200 includes two or more valve openings and, as shown in the present example, includes four such openings 201 to 204, each of a different size from one another. Typically, the openings 201 to 204 have a diameter at their narrower end of 5 mm, 7 mm, 10 mm and 12 mm. The openings 201 to 204 are of the same general kind used in conventional therapy devices having a non-linear orifice with a generally trumpet shape arranged with their wider side facing upwardly towards the rocker arm 106. The valve members or openings 201 to 204 are evenly spaced around a rotatable circular support 205 so that each is spaced from its adjacent openings by 90°. The support 205 is rotatable about a vertical axis, that is, an axis orthogonal to the longitudinal axis of the device 100, between four different positions where different ones of the openings 201 to 204 are positioned directly below the conical valve closure 107 on the rocker arm 106. The support 205 is rotated by means of a ribbed scroll wheel 206 extending externally around the support and accessible either directly or indirectly, such as via an intermediate gear wheel, through a slot 207 in the hinged cover 102 of the housing 101. The device 100 includes some means for lifting the rocker arm 106 to enable the support 205 to be rotated. This may take the form of a cam on the scroll wheel 206 that engages the rocker arm 106 when the wheel is rotated.

The device also includes a magnet arrangement comprising a movable magnet structure 210 arranged to interact with the soft-iron pin 108 mounted on the rocker arm. The structure 210 comprises a circular turntable 211 supporting two small permanent magnets 212 and 213 mounted diametrically opposite one another and having different magnetic energy products. The turntable 211 is rotatable about a vertical axis so that a selected one of the magnets 212 or 213 can be positioned adjacent the underside of the soft-iron pin 108 on the rocker arm 106. In this way, the magnetic force pulling the free end of the rocker arm 106 down and urging the selected valve opening 201 to 204 closed can be adjusted between two different values. The turntable 211 also supports an external scroll wheel 220 extending around the turntable and that can be manually displaced through the slot 207 in the hinged cover 102. Alternatively, the valve support and magnet support could be arranged such that they could only be accessed after having opened the hinged cover 102. There are many other arrangements by which the valve openings could be selectably connected in the breathing path through the device, such as on a linearly displaceable slider. Similarly, the magnet arrangement need not be rotatable but could, for example, be slidable. The valve openings 201 to 204 need not be of the trumpet shape with a non-linear profile but could be of other shapes.

It can be seen, therefore, that when the user inhales via the mouthpiece 103 and opening 104 air is drawn through the outlet 111 and the one-way valve 112 into the passage 110. The reduced pressure this creates in the passage 110 applies a pressure tending to draw the rocker system 105 further down against the valve seat 200, thereby further enhancing the normally closed state of this valve. When the user exhales through the mouthpiece 103 and inlet 104 this creates an increased pressure in the passage 110, which is prevented from flowing directly to the outlet 111 by the one-way valve 112. Instead, the pressure causes the valve closure 107 in the magnetic rocker system 105 to open, momentarily lifting the rocker arm 106 and then allowing the pressure below the valve to drop. This allows the rocker arm 106 to fall and close the valve seat 200 again until pressure builds up sufficiently to lift the rocker arm. In this way, the valve mechanism 105 alternately opens and closes, causing an oscillation or vibration in the air flow along the passage 110. This oscillation communicates with the user's respiratory system to produce a therapeutic vibration that helps loosen secretions.

The device described has several advantages. It reduces the need to manufacture and stock devices with different workflows thereby enabling the cost of manufacture and the cost to the user to be reduced. It also enables the user to be provided with a single device that can be adapted to meet the user's change in respiratory function. In this way the user obtains maximum benefit.

1-7. (canceled)

8. A respiratory therapy device having an inlet through which a user breathes, an opening to atmosphere, and a valve mechanism driven by breathing through the device to produce an oscillating resistance to breathing, characterised in that the valve mechanism includes at least two valve members of different sizes that are selectably connected in the breathing path through the device such that the patient can breathe through one or other of the valve members.

9. The respiratory therapy device according to claim 8, characterised in that the valve mechanism includes a common valve closure, and that the valve members have openings of different sizes that can be selectively aligned with the valve closure.

10. The respiratory therapy device according to claim 9, characterised in that the valve members are mounted on a support that is displaceable relative to the valve closure.

11. The respiratory therapy device according to claim 10, characterised in that the support is rotatable about an axis parallel to the flow path through the valve members.

12. The respiratory therapy device according to claim 9, characterised in that the device includes a magnet arrangement for providing a restoring force to the valve closure tending to urge the valve closure to a closed position.

13. The respiratory therapy device according to claim 12, characterised in that the magnet arrangement includes at least two permanent magnets of different strength that are selectively displaceable into magnetic interaction with a magnetic element on the valve closure.

14. The respiratory therapy device according to claim 13, characterised in that the permanent magnets are mounted on a turntable, and that the turntable is rotatable about an axis parallel to the flow path through the valve members.
